(19) United States
(12) Patent Application Publication
(10) Pub. No.: US 2011/0009708 A1
(43) Pub. Date: Jan. 13, 2011

(54) System and apparatus for improved nutrition analysis

(76) Inventor: Jeffery Boyes, San Diego, CA (US)

Correspondence Address: Bergman & Song LLP, P.O. Box 400198, Cambridge, MA, 02144, US

(21) Appl. No.: 12/803,620

(22) Filed: Jun. 30, 2010

Related U.S. Application Data
(60) Provisional application No. 61/269,888, filed on Jun. 30, 2009.

(51) Int. Cl. A61B 5/00

(52) U.S. Cl. 600/300

(57) ABSTRACT

A system having an incorporated software program attempts to tailor a person's diet with the person's desired nutritional goal and their current food consumption.

### CROSS REFERENCE TO RELATED APPLICATION

This application claims the benefit of U.S. Provisional Patent Application No. 61/269,888, filed Jun. 30, 2009, the disclosure of which is incorporated herein by reference in its entirety.

### BACKGROUND OF THE INVENTION

The invention is related to the field of nutrition and physiology and more specifically to improved personal nutritional physiology awareness and programming, through applied nutrition and analysis.

Over the years, the investigation of human physiology has significantly increased. Global research has resulted in an increased knowledge of various aspects of the functionality of the human body. Part of this knowledge includes functional physiological limitations. Additional knowledge includes human “best practices” for growth, strengthening, and recovery/repair.

Over the past many years, there has also been an increased desire to for individuals to take better care of themselves. This increased sensitivity creates a need for increased knowledge about their bodies and themselves. This need translates into an increased need for nutrition education. Ideally, the education is tailored to the individual's needs as well as the individual's ability to comprehend in the information.

The primary limitations of the media tools currently available are the practical application of nutritional principles and theories and the time-consuming efforts required to sorts through the databases or programs in hopes of finding the information desired. Both factors demonstrate an increased need for educational tools that provide credible information in nutrition that is easily accessible and interpreted by health professionals.

This increased need for nutrition education has resulted in the development of many media tools and resources utilizing current advancements in technology. These primarily include software programs and, within the last decade, the increased appearance of websites on the Internet focusing on less than adequate nutrition information. An intent of these services are to “narrow the gap” between research and application. However, this is ultimately where the resources are currently inadequate. Individuals most interested in applying the knowledge gained from the studies are not catered to with regards to the layout and interpretation of information.

### BRIEF DESCRIPTION OF THE DRAWINGS

FIG. 1 is a computer system in accordance with an exemplary embodiment of the invention;

FIG. 2 is program logic diagram of the computer system of FIG. 1;

FIG. 3 is a logic flow representing an aspect of the computer system of FIG. 1 in greater detail;

FIG. 4 is a representational diagram depicting an aspect of the computer system of FIG. 1;

FIGS. 5(a), (b) are representational diagrams depicting an aspect of the computer system of FIG. 4 in greater detail;

FIG. 6 is a representational diagram depicting an aspect of the computer system of FIG. 4 in greater detail;

FIG. 7 is a representational diagram depicting an aspect of the computer system of FIG. 4 in greater detail;

FIGS. 8(a), (b), (c) are a representational diagrams depicting an aspect of the computer system of FIG. 1;

FIG. 9 depicts a screen shot of a computer display running the computer system of FIG. 1;

FIG. 10 depicts a second screen shot of a computer display running the computer system of FIG. 1; and

FIG. 11 is a logic flow representing another aspect of the computer system of FIG. 1.

### DETAILED DESCRIPTION OF THE INVENTION

In the following detailed description, reference is made to the accompanying drawings, which form a part hereof, and in which is shown by way of illustration specific exemplary embodiments of the invention. These embodiments are described in sufficient detail to enable those of ordinary skill in the art to make and use the invention, and it is to be understood that structural, logical, or other changes may be made to the specific embodiments disclosed without departing from the spirit and scope of the present invention.

The invention seeks to address a deficiency exists in nutrition advising and applications for working health professionals. Today thousands of professionals whose jobs include researching, consulting, or teaching nutrition information must sift through an abundance of educational materials in order to locate desired information. The majority of these persons have educations ranging from certifications to graduate degrees in related fields. However, the predicament isn't whether they are qualified or have the appropriate education to fulfill their work requirements. Rather it is that their services are very much inefficient because a single, effective nutrition advising source doesn't exist. The invention addresses the problems identified above as the invention seeks to narrow the gap between research and the application and/or distribution of nutritional information. The invention applies knowledge acquired from various studies and tailors it, with regards to the layout and interpretation of information, to an individual's needs through the use of a software program on a computer system.

Currently nutritional educational strategies derived from studies on human physiology and metabolism are used to enhance a person's health and athletic performance. Generally, the educational information is conveyed through the distribution of hard copies, e.g. texts, journals, and magazines.

Arguably, there exists more than a casual connection between a health care professional's assimilation of available educational information and the professional's ability to apply the information. The more digestible the information, the more likely that the information can be applied. It is not only the presence of this information but, more importantly, the understanding and interpretation of the literature that allows health and human performance professionals to accurately and appropriately apply learned information to an individual.

An exemplary nutritional advising system will include several functions including a nutritional analysis and advising tool and an information resource tool.

A first function of the system is a nutritional analysis and advising tool: a calculation of an individual's, e.g., an athlete's, nutrient requirements and explanation of methods of applying the information to the individual's lifestyle. In addition to the display of his/her diet requirements, the system will have fields where the individual can input his/her diet and supplementation, in order to have their current dietary practices analyzed. This is optimal for the determination of the individual's metabolic and nutritional requirements.

A second function of the system is informational/educational and is a database that provides connections, e.g., Internet links, to health and nutrition articles that provides educational resources to a multitude of nutrition and related sports science practices. The articles will be written and reviewed by professionals who will add substantial credibility to the system. The education portion of the system will not only be limited to standard literature articles, but will also have the capability of educating through non-traditional means; such as, education videos, video conferencing, and Q & A sessions with various professionals in the field of sports and sports nutrition. A system according the invention provides a professional with a single source, e.g., access point, for educational material such as articles, studies, and other educational tools. The invention provides increased ability for the professional to access the materials, and reduces the likelihood that the professional will experience research failure or fatigue resulting from having to search for educational material. Thus the system will serve as an encyclopedia of information surrounding nutrition and physiology. The search component of the site will allow the professional to easily navigate throughout the archives to find the desired information. Utilizing both software and the internet allows for endless capabilities for any application, because of functionality and diversity with regards to the types of information that can be provided. The professional will benefit most from the program because of ease of use and accessibility to a plethora of knowledge.

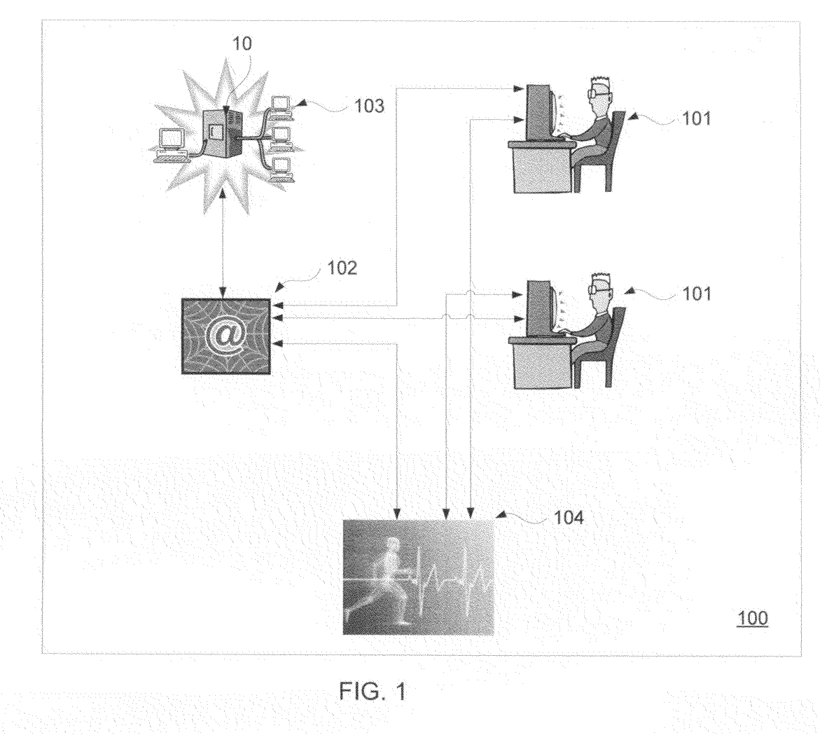

FIG. 1 depicts a system 100 that incorporates a nutritional advising software program 10 in accordance with an exemplary embodiment of the invention. System 100 includes a processing program 103 connected through a network 102, e.g., the Internet 102, to a computer 101. Although described in reference to a computer networked to a server, the computer is not limited to being a laptop computer, desktop computer or a dumb terminal, and can also include personal digital assistants (PDAs), smart phones, and similar type computing mechanisms). The processing program 103 is a computer processing system or server that stores and executes the program coupled to the Internet 102. The processing program 103 can include other computers connected via a network, e.g., an intranet network. A user accesses the program 10 through a computer terminal 101 and executes the program. The program 10 resides on the processing program 103; alternatively, the software program 10 resides in part, or in whole, on a standalone computer 101. In one aspect, databases used by the software program 10 can also reside on the same computer 101 where the software program 10.

An exemplary embodiment is a program incorporated into a computer system that provides tools for nutritional analysis and advising based on health and human performance information. In an aspect, the program provides nutritional advising tailored for the needs of a person.

There are three types of persons that will use the system: a professional, a client, and an individual. A professional is generally a health care professional who is to some degree responsible for the welfare of the person, e.g., a client. Typical professionals are health care professionals, which includes sports trainers, coaches, dieticians, and physical therapists. Ideal professionals of the system include health-care and human performance professionals, and are not limited to dietitians/nutritionists, conditioning coaches, athletes, personal/athletic trainers, physicians, holistic health-care practitioners, and chiropractors. This does not limit a person who is not a health-care or human performance professional from using and gaining benefits of the system.

Clients are persons who are seeking nutritional guidance and most typically, but not exclusively, are athletes, professional, amateur, scholastic and weekend warriors. Clients are typically associated with a professional, who works with the client to maintain or improve his/her nutritional intake and athletic performance. The invention receives a client's personal physiological characteristics and the client's goals and the professional uses the invention to apply the characteristics and goals to provide nutritional guidelines, including, but limited to, providing a nutritional plan, e.g., a meal plan, for the client. The invention also provides the professional with tools to analyze the client's diet and client's measurable abilities.

Individuals are persons who are seeking nutritional guidance and most typically, but not exclusively, are athletes, both professional and weekend warriors. In a preferred embodiment, individuals are different from clients in that individuals, for the purposes of the invention, are not associated with a professional. Thus, the individual performs his/her own analysis. The individual uses the invention to apply his/her own personal physiological characteristics and goals to provide nutritional guidelines, including, but limited to, providing a nutritional plan, e.g., a meal plan, for himself/herself. The invention also provides the individual with tools to analyze the his/her diet and measurable abilities.

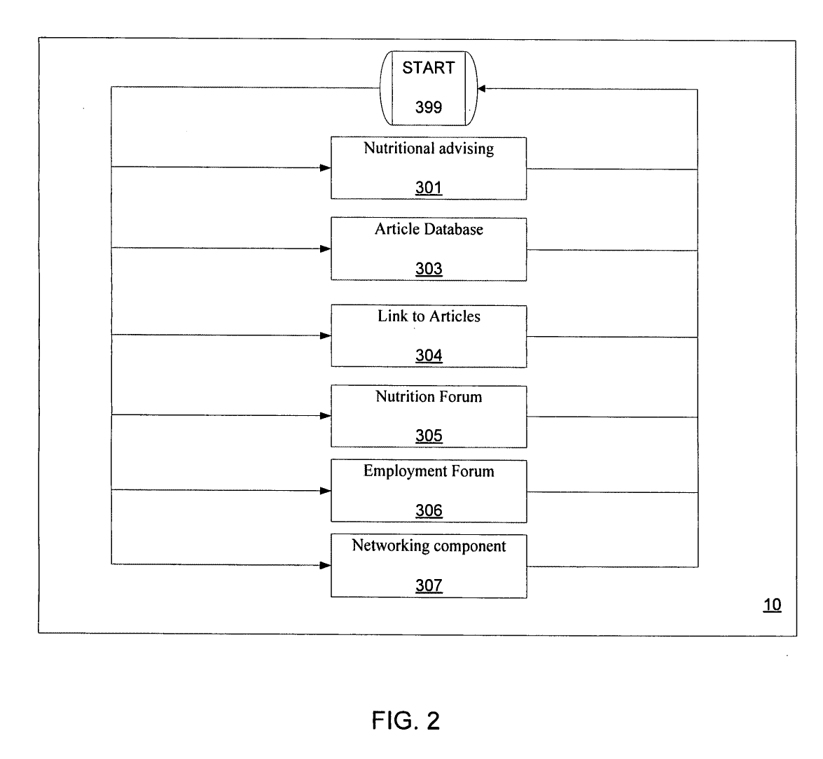

The program 10, e.g., a program on a networked computer system 100, preferably networked to resources in the Internet, allows for the creation of the following components, segments, or sub-programs, of the program as depicted with respect to FIG. 2.

FIG. 2 is a system logic diagram of components of the program 10 incorporated in the computer system 100 of FIG. 1 according to an exemplary embodiment. The program 10 includes: a nutritional advising component 301, an article database component 303, a link to articles component 304, a nutritional forum component 305, an employment forum component 306, and a professional networking component 307.

1. Nutritional Advising component 301: Nutrition advising is based on inputting an individual's characteristics, both physical and lifestyle, in order to determine nutrient requirements based on a selected goal. Exemplary nutritional goals, which also may include physiological goals, are discussed in greater detail below. Selecting this segment program execution continues to segment 499, e.g., a home page of the user.

2. Database of Nutrition Articles component 303: In an exemplary embodiment, this component eliminates the numerous inefficient hours used to research a given nutrition topic, and would include articles written by both graduate/doctoral students and well-respected professionals in various fields of human science. This component provides the ability to access a central location for the posting and viewing of nutrition articles. The articles can be searched by title, subject, author, category, and other relevant data. The articles can be browsed, by most recent, least recent, most viewed, least viewed, most favored and least favored. The articles can also be view by date, for example, the system displays only those articles from: today; this month; or this year. The articles can be downloaded and printed. When done with this component, program execution returns to segment 499.

3. Link to Scientific Research Articles component 304: Thousands of articles are published each year in nutrition-related fields. This is the foundation for all literature and education produced describing the outcomes, ranging from magazine articles to university textbooks. They are available to view, however, the majority of professionals does not have the knowledge or time to locate the research and determine its applicability. This component provides the ability to access a central location for the posting and viewing of scientific research articles. The articles can be searched by title, subject, author, category, and other relevant data. The articles can be browsed, by most recent, least recent, most viewed, least viewed, most favored and least favored. The articles can also be view by date, for example, the system displays only those articles from: today; this month; or this year. The articles can be downloaded and printed. When done with this component, program execution returns to segment 499.

4. Nutrition Forums component 305: Currently a single, credible nutrition forum does not exist that allows for an inter-exchange of information from healthcare professionals, students, coaches, and scholars. The responses in the various Q & A sections will be cited and the author's credentials will be displayed, with additional capabilities to communicate directly with involved professionals if further assistance is needed or knowledge desired. In this component, forum topics are provided to a user. The user can browse forum topics or can search forums by subject, keyword or time.

The forums can be browsed, by most recent, least recent, most viewed, least viewed, most favored and least favored. The forums can also be viewed by date, for example, the system displays only those forums from: today; this month; or this year. The forums can be downloaded and printed. When done with this component, program execution returns to segment 499.

5. Employment/Resume forum component 306: Currently there are numerous employment sites that healthcare professionals have to search in order to possible locate an employment opportunity in his/her field. By including this function, an embodiment of the invention allows professionals to post their resumes, with direct links to any articles authored within the site, and search job opportunities posted by professional groups or companies. When done with this component, program execution returns to segment 499.

6. Professional networking component 307. The program 10 also provides a resource where system professionals can network with other system professionals. In this component, communities are established, which system users can subscribe to. A user can view others in the community and enables communication through email or, IMs, or mobile phone, etc, to others in the community. Users can also be viewed by date, for example, the system displays only those users from: today; this month; or this year. Users can also be searched by community and by when the other user's information was modified. The user's information can be downloaded and printed. When done with this component, program execution returns to segment 499.

Nutritional advising program segment 301. As part of an exemplary system 10, the system is programmed with at least one nutritional goal. Exemplary nutritional goals include: standard fitness, sports, fitness, holistic, and medical. A “Standard nutrition” goal is directed to a person who has a sedentary and minimally active life style but also is directed to a person who wants to maintain a fit lifestyle; a “sports nutrition” goal is directed to a person who wants to establish a diet in order to optimize athletic ability; a “holistic nutrition” goal is directed to a person who seeks to maintain a holistic approach to his/her diet; and a “medical nutrition” goal is directed to a person who has an active life style and to a person with a health condition that is, or can be, directly affected by his/her diet.

With a standard nutrition goal, ideally, there are minimal changes to an individual's daily caloric or macronutrient (protein, carbohydrate, fat) percentages or ratios. Adjustments concentrate on a more balanced approach to health and well being for the purposes of altering some component of the individual's health characteristics (general health and well-being). For example, with the goal of standard nutrition the program 301 is programmed to provide the nutritional information for the individual to focus on the effects of food intake on his body. The effects are provided by displaying a nutritional breakdown of his daily food intake. For example, the program displays the user's daily calories, carbohydrates, proteins, and fats. When using standard nutrition goal in an exemplary system, the individual chooses a specific goal: adjust weight (lose or gain), adjust body fat percentage (decrease or increase body fat percentage), and adjust muscle mass (decrease or increase muscle mass percentage). Program 301 is dynamic and is capable of including additional nutritional goals.

The program 301 provides guidance to a user to create a meal plan, i.e., a diet, based on the goal selected by the individual or client. If an individual or client does not select a goal, then a default goal, which is predefined by an administrator of the program 301, is utilized, e.g., standard nutrition. An individual or client is not limited to a single goal and at different times can select different goals. For example, at a first time, an individual or client selects the goal of standard nutrition and also selects weight loss as a first specific goal. At a later time, the individual or client selects the goal of medical nutrition. An individual's or client's selection of a nutritional goal for himself/herself does not affect the ability of any other individual or client who is also part of the program 301, to select a nutrition goal.

The program 301 analyses an individual's or client's estimated food intake, compares it to the individual's or client's desired goal and provides goal oriented nutritional information. For example, an individual or client seeks to lose weight and therefore selects her/his goal as standard nutrition, and specifically, lose weight. In an approach program 301 has the individual or client define how much weight the individual or client would like to lose and the desired goal date. Program 301 uses the individual's or client's personal characteristics and the time period to reach the goal weight to determine a projected rate of weight loss and calculates the recommended daily nutritional intake to reach this goal, e.g., the rate of loss. The nutritional intake is the nutritional value(s) of the food to be consumed by the individual or client. The nutritional values include, for example, the KCalories, proteins, fats, and carbohydrates of the food.

The recommended daily nutritional intake is the daily total of the nutritional value(s) of the food to be consumed by the individual or client, for example, the daily total of KCalories, proteins, fats, carbohydrates, minerals and vitamins of the food consumed that day. The program 301 compares this recommended daily nutritional intake to nutritional guidelines to confirm that the rate of loss is within recommended guidelines. The nutritional guidelines are those, for example, established by the United States Department of Agriculture and stored in, or accessed by, program 301. In another aspect, an administrator of the program 301 defines nutritional guidelines for users of the program 301. The program 301 provides the KCalories, proteins, fats, and carbohydrates of the food to the individual or client as the individual or client performs his meal planning.

In another aspect, the program 301 uses the individual's or client's personal characteristics, energy expenditure, and the time period to reach the goal weight to determine a projected rate of weight loss and calculates the recommended daily nutritional intake to reach this goal.

The system also provides a “sports nutrition,” i.e., the system is programmed to provide the nutritional information for the individual or client to maintain a nutritional needs for someone who actively participates in sports. In one aspect, it is a nutritional goal for an individual or client who is an athlete to optimally perform at his/her sporting event or training regimen. In an aspect, the recommended daily nutritional intake is more closely tied to the individual's or client's activities. The program 301 analyzes the individual's or client's personal characteristics and the estimated energy expenditure, and calculates the recommended daily nutritional intake to improved performance through better nutritional intake. In an exemplary embodiment, the system assists in planning meals for a day which includes planning for pre, during, and post training nutrition, e.g., nutrition within, before and after, one to two hours of training, and nutrition during training.

In this aspect, the system may incorporate an individual's or client's nutritional needs based on the training context. The training context also factors into nutritional intake, for example, the training season or competition season when the meal will take place: a time period prior to or after a competition, or during off-season, pre-season, in-season, and post-season training. Each context may suggest different nutritional intake. For example, during off-season, a higher percentage of calories may be attributable to protein intake. Additional program capabilities include associating the individual or client with a specific sport and season or time period (e.g. pre-season & football), and the nutritional recommendations are tailored specifically for the individual or user according to the sport and time period. The recommended nutritional guidelines are derived from research that has identified specific nutritional needs for a football player prior to the season starting. This is an example of an application of current & advanced research in sports nutrition and exercise physiology.

With a “medical nutrition” goal, program 301 guides an individual or client to create a diet plan based on the individual's or client's medical pathology(ies), Nutrition prescription and information on dietary practices for medically diagnosed pathologies (e.g., diabetes, crohn's disease, organ malfunction . . . ). For example, if the individual or client has high cholesterol issues, the program 301 identifies foods selected by the individual or client that could negatively impact the individual's or client's cholesterol, i.e., foods having high cholesterol levels or that are high in saturated fats.

When a “holistic nutrition” goal is selected, program 301 guides the individual or client to create a diet plan based on the individual's or client's general or specific holistic guidelines. With an emphasis on quality ingredients, a holistic diet consists of organic whole foods that are generally grown locally and eaten while still in-season. Holistic eating focuses on incorporating foods that are nutrient-dense while avoiding foods that have been depleted of their nutrient content, such as processed foods or foods that have been degraded during growth and/or preparation.

The program 301 is programmed with nutritional and physiological guidelines. For example, the program 301 incorporates the United States Department of Agriculture's established guidelines; which include, for example, recommended daily intake and allowances of: protein, various fats, and simple and complex carbohydrates. Alternatively, the guidelines include the recommended daily intake and allowances of: fruits, grains, vegetables, milk, and meats. The exemplary program 301 also includes guidelines from the American Dietetics Association (ADA), the American Heart Association (AHA), the American College of Sports Medicine, the International Society of Sports Nutrition (ISSN), the American Diabetes Association, the American Society for Nutrition (nutrition.org), the National Institute of Health (NIH), and the World Health Organization (WHO).

In an exemplary embodiment, the program 301 is dynamic and is designed to incorporate recent developments and advances in nutritional and physiological guidelines. Thus, recommended daily nutritional intake levels are modifiable for each individual and client. Furthermore, additional nutrients can be added to the system for tracking. For example, a specific enzyme can be added to be tracked along with, or in place of other nutritional values. For example, a study conducted in the future could determine that significantly reducing carbohydrates from an individual's daily intake increases, in the short term, the elimination of body fat. A system administrator can include this result in the system programming as a guideline to an individual seeking to reduce body fat. In an aspect, a system administrator or a professional can modify an individual's or client's recommended carbohydrate intake and significantly reduce it. While meal planning, the individual or professional will utilize this recommended carbohydrate intake when selecting foods.

Although the program 10 is described as being a software program, it is not limited to software and can be programmed in hardware, or in a combination of hardware and software. Furthermore, although the system 100 is described as using the Internet as a networking mechanism, the system 100 is not so limited and may use any other networking system. Furthermore, the system 100 can be implemented on a standalone program 10, e.g., a personal computer with limited or no networking to other computers. The system 100 can also be implemented as an application on a smart phone or PDA. When a system record, e.g., a professional's record or an individual's record, is modified, the new information is saved and a date is associated with the change. The old record information is also saved. The date is associated with the entry, either by system default or manual entry for tracking the appropriate date that record information was recorded/noted.

In the Nutritional Analysis program segment, in an exemplary processing flow, the user utilizing the program 301 will have multiple options and can systematically navigate them to retrieve desired information. Exemplary logic flows of two nutritional planning options, e.g., “Standard Nutrition” and “Sports Nutrition” are described below.

Professionals and athletes that have access to the resources of the system will have the capability of accessing accurate and credible information regarding the application of sports nutrition principles. The information will be displayed in several contexts, in order to simplify the explanation and application of the information, including but not limited to, individual assessments, online video tutorials, detailed graphs and charts.

An exemplary system 100 has three types of users: professionals, clients, and individuals. Professionals are provided the capability to create a client(s) profile or a team(s) profile (i.e., a group of clients identified by the professional), generally his/her clients, as users of system 10. With respect to an exemplary program 301, a professional can: enter client information for a client, enter a client's characteristics for the client, enter a client's food preferences for the client, enter the client's energy expenditure for the client, enter the client's food intake for the client, enter the client's nutritional goal for the client, display a summary of the client's status, perform exercise prescription, perform meal planning, upload images of the client, and perform analysis of the data in a client's profile. The professional can also do meal planning and analysis for a group. For example, a professional plans meals for all of the members of the local football team as single plan and has the plan provided to all of the members of the team. The professional can perform analysis of a group. For example, the professional tracks carbohydrate consumption by the members of a team. Thus, the professional's capabilities include inputting and tracking an individual client's or a team client's health, e.g., nutritional, and sports, e.g., performance, factors, creating and managing nutrition of a client's or team, and providing educational materials and communication with their clients or other individuals. The professional can also act on behalf of client and enter information into a client's record in the system 100. For example, a professional can enter a client's characteristics, food intake, or food preferences.

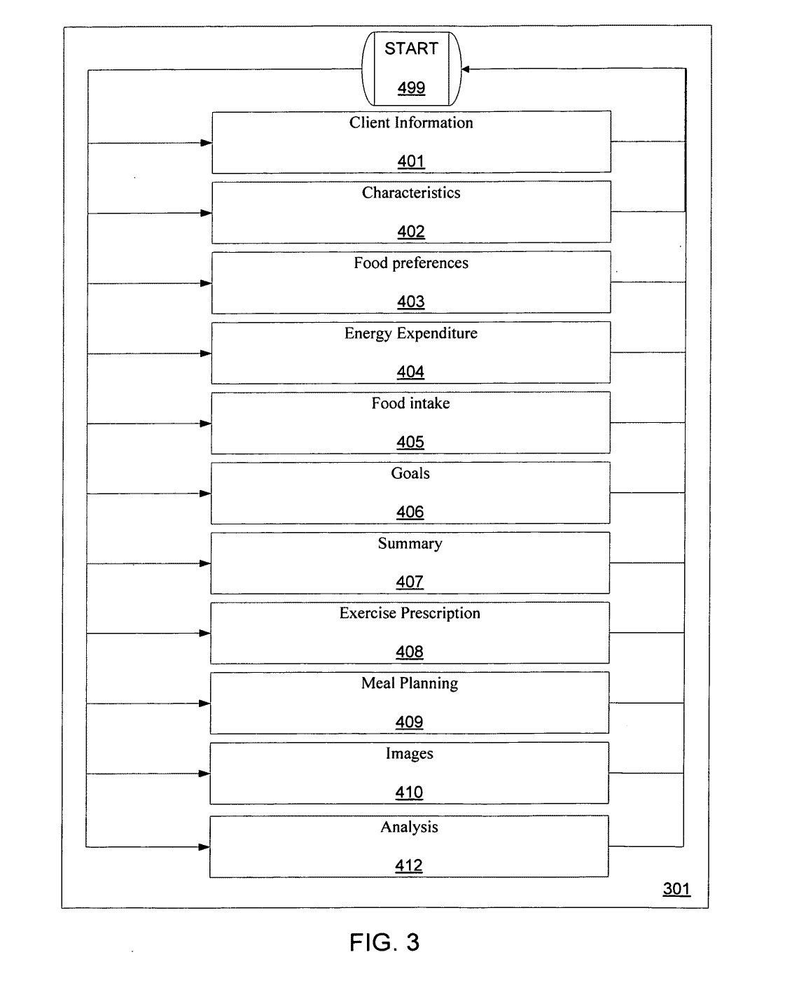

An individual is provided most of the capabilities of a professional described above with regards to nutrition programming but is limited to accessing and analyzing only his or her profile in the system 100. Thus, an individual cannot access a third party's profiles. With respect to an exemplary program 301, an individual can: enter his/her client information, enter a his/her characteristics, enter his/her food preferences, enter his/her energy expenditure, enter his/her food intake, enter his/her nutritional goal, display a summary of the his/her status, perform meal planning, upload his/her images of the client, and perform analysis on data in his/her profile. See, for example, FIG. 3, described below.

The client is provided most of the capabilities of an individual as described above with regards to nutrition programming but is limited to accessing and analyzing only their profile in the system 100 and cannot access the third party's profiles. The client cannot perform meal planning. With respect to an exemplary program 301, a client can: enter his/her client information, enter a his/her characteristics, enter his/her food preferences, enter his/her energy expenditure, enter his/her food intake, enter his/her nutritional goal, display a summary of the his/her status, upload his/her images of the client, and perform analysis on data in his/her profile. See, for example, FIG. 3, described below.

FIG. 3 is a logic flow representing an aspect of the program 10 of FIG. 1 depicting the standard nutritional analysis component 301. As such, the system logic flow described below is for the Standard Nutrition option according to an aspect of the invention. A program user, e.g., a professional, individual, and client, only has access to program segments of 301 as noted above dependent on the type of user.

Initially, a program user is provided several program flow options which permits the user to add or modify data in her/his profile, or in the case of a professional, his/her client's profile. Starting from “Start 499”, a user can choose the program to flow to any of the following segments that are permissible as defined by the type of user: client information 401, characteristics 402, food preferences 403, energy expenditure 404, food intake 405, goals 406, summary 407, exercise prescription 408, meal planning 409, images 410, and analysis 411. In an exemplary approach, “Start 499” is a home page of an individual or user that is displayed after the individual or user has properly logged into the system 100 with a proper name and password.

For a nutritional analysis to provide reasonable results, certain information should be entered before executing other segments. For example, a client's or individual's characteristics should be entered before executing any other segment. Furthermore, a professional or a system administrator must create a system profile for an individual or client before the user can access and add any information to the profile in the program 10. When the professional or administrator adds an individual or client, the program 10 creates a new profile, e.g., a database record, for the individual or client. The professional generally populates the profile with limited information, typically the individual's or client's name, and the remaining data fields of the profile remains empty.

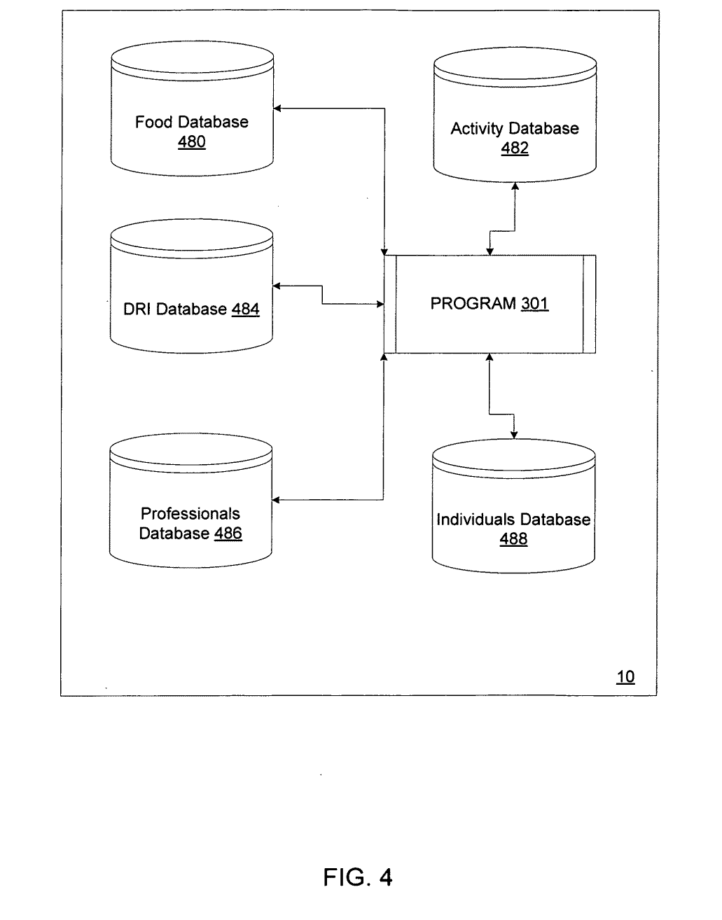

In an exemplary approach, the program 301 as part of program 10 includes and/or accesses several different data sources: a DRI database 484, an activity database 482, a food database 480, an individuals database 488, and a professionals database 486 as depicted in FIG. 4.

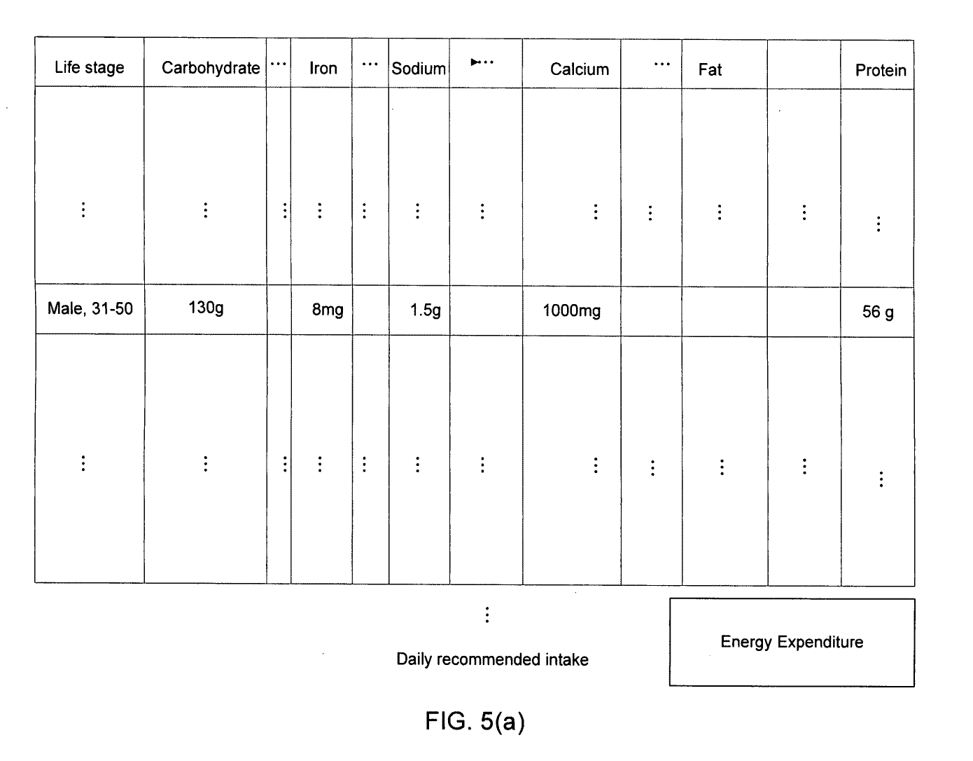

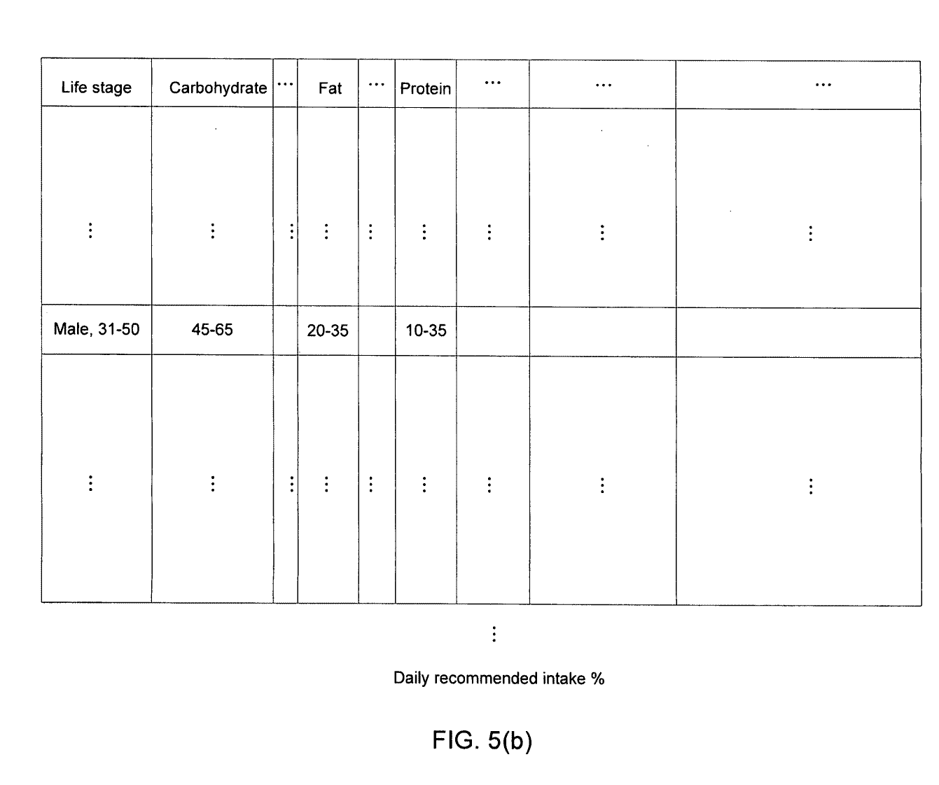

The DRI Database 484. This is a database of the daily recommended intake (“DRI”) of several dietary nutritional components. An exemplary embodiment, the DRI database 484 includes the daily recommended intake for different dietary components. An example of the database is depicted in FIGS. 5(a) and 5(b) which represents a plurality of data records of the recommended daily nutritional intake. For each age group and gender there is an associated value that either reflects the desirable percentage (%) of the total calories for the day or the desirable amount (e.g., grams, milligrams, micrograms, etc) consumed during the day of the nutritional component, although only one age group and gender is actually depicted. The nutritional components generally include, for example, various electrolytes and water, elements, macronutrients, and vitamins, and generally include specific descriptions of the components: carbohydrates, fiber, fat, fatty acids (which can be further specified into various saturated and unsaturated fats), proteins, amino acids, minerals, sodium, potassium, water, and vitamins. In an exemplary approach, the DRI database 484 may also include recommended maximum and minimum values of the nutritional components.

In an aspect, the DRI database 484 also includes a table of the estimated energy expenditures based on a person's gender, activity level, age and body mass index, which provides an estimated daily caloric intake to match the estimated energy expenditures. If the individual or client is a woman who is pregnant or lactating, then the DRI database 484 may have different values for their daily recommended intake (“DRI”). Program 301 uses values of the dietary components from the DRI database 484 to determine an individual's or client's recommended daily nutritional intake as part of the individual's or client's nutritional planning. In an exemplary aspect, the contents of the DRI database 484 can be modified to provide additional or remove nutritional components and to modify the nutritional components and the associated values that exist in the DRI database 484.

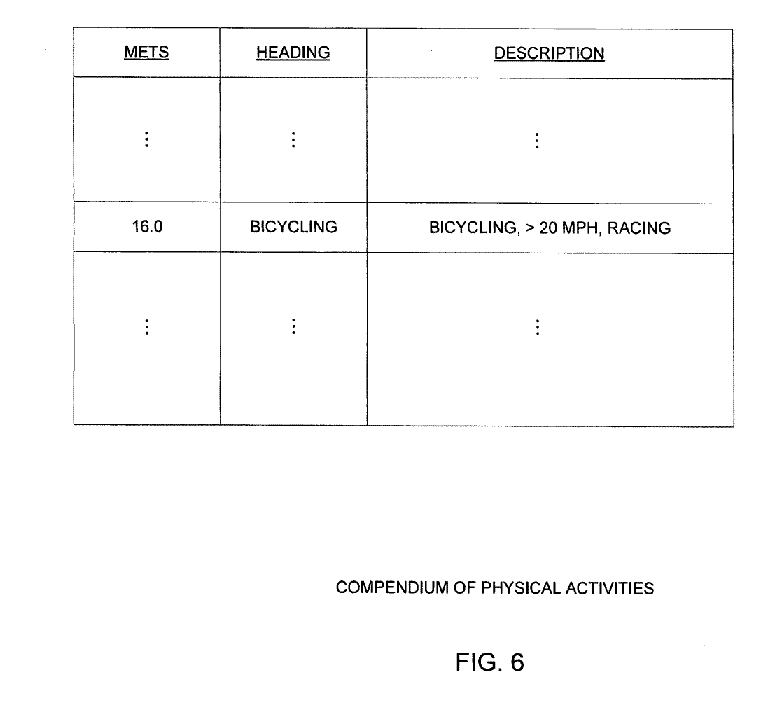

The Activity Database 482 is a compendium of physical activities. In an exemplary approach, the Activity Database 482 includes a list of at least 900 different activities which is broken down by at least description, heading, and METS. For example, FIG. 6 depicts a database of activities and the corresponding MET value, although only one entry is shown. Program 301 uses the list of activities from the activity database 484 to assist the individual or user in describing his/her energy expenditure. Program 301 uses the MET value associated with the activity to determine an individual's or client's energy expenditure. In an exemplary aspect, the contents of the activity database 482 can be modified to provide additional or remove activities and to modify the activities and the associated values that exist in the activity database 482.

The Food database 480 is a compendium of food. Each food is either a food that can be eaten, e.g., an egg, a banana, commercial cereal, or it is a food that is prepared, thus the food database 480 includes the recipe for the food. In an exemplary approach, food database 480 includes a list of at least 40,000 different foods which provided as part of pre-defined foods for the program 10. Each of the food items in the database is further described in terms of a corresponding category(ies) and its Nutritional components for a defined serving size, e.g., one-half cup of the food. In an exemplary approach, food is associated with at least one category. In an exemplary approach, the Nutritional components include the food's calories, fats, carbohydrates, and proteins, which maybe further broken down into more specific components. For example, carbohydrates are further described in terms of simple and complex carbohydrates. Additional components may include, but are not limited to, total cholesterol, amino acids, different vitamins, and different minerals.

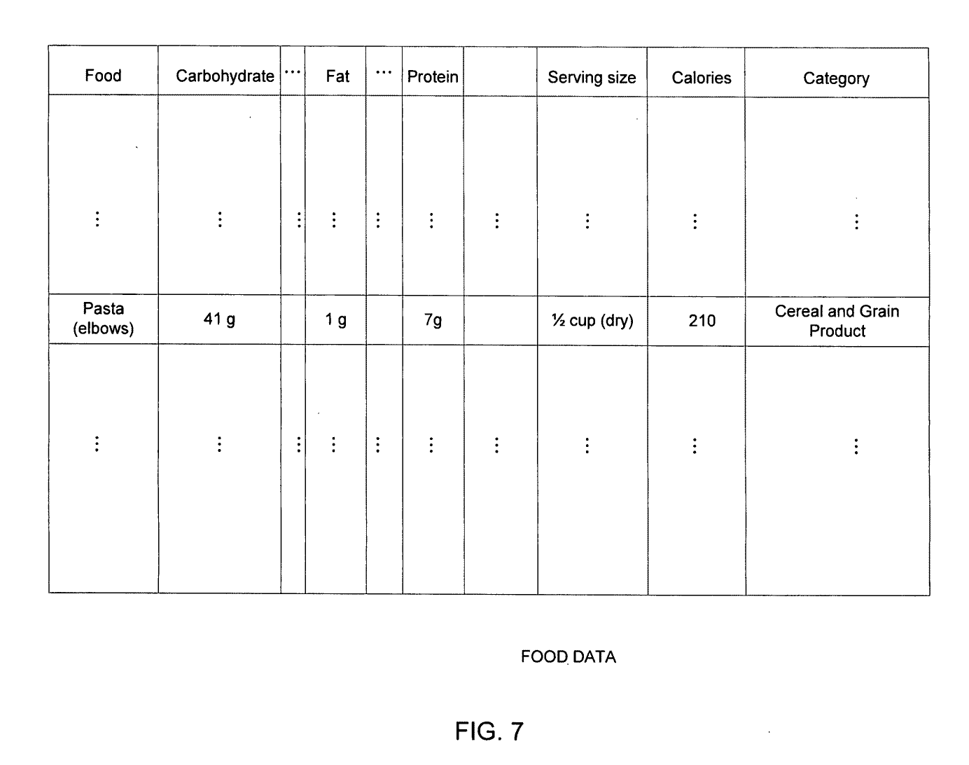

Each food that requires preparation includes a recipe that lists the constituent foods, the nutritional components of each constituent food and the total nutritional components for the recipe, i.e., the sum of all of the constituent foods' nutritional components. The food database 480 preferably also includes recipe instructions. FIG. 7 depicts a food database, although only one entry is actually shown. As seen in FIG. 7, the food is pasta (elbows), which has 41 grams of carbohydrates, 1 grams of fat, 7 grams of protein, and 210 calories for each serving size of ½ cup of dry elbows.

Program 301 uses the food and its associated nutritional components of food database 480 in meal planning, food intake, identifying an individual's food preference(s) and when adding foods and recipes for food to food database 480. The food database 480 is appendable by users of the program 301. Users can add a food and provide the category and the nutritional components for a new or existing food. Users can add a recipe and provide the category, provide the constituent foods, the amount of each of the constituent foods, the nutritional components for each the foods, and the total of each of the nutritional components for the recipe. The individuals database 488 or the professionals database 486 records who adds the food or recipe depending on the user who adds the food. If the user is a professional, then the profile of the professional in the professionals database 486 notes that the food he/she added. If the user is an individual or a client, then the profile of the individual or client, respectively, in the individuals database 488 notes that the food he/she added.

The individuals database 488 contains the profiles for each of the individuals and clients of the system 100. In an exemplary aspect, for each individual and client, a record is kept which includes, at least: identifying information about the individual or client, food preferences, her/his system privileges, her/his meal plans, her/his activity records, her/his meal prescriptions, and her/his activity prescriptions. The professionals database contains the profile for each of the professionals of the system 100. In an exemplary aspect, for each professional, a profile is kept which includes, at least: identifying information about the professional, her/his system privileges, her/his list of individuals who are clients and her/his list of teams who are clients.

Referring again to FIG. 3, a user selects individual's information 401 and the program 10 execution will continue to this program segment. In this segment, the user enters biographical information of a client or individual. In an exemplary aspect, the biographical information includes the individual's or user's contact information, e.g., address, phone, e-mail. When completed, program execution returns to segment 499.

From segment 499, a user selects characteristics 402 and the program 10 execution will continue to this program segment. In this segment, an individual's or client's physiological characteristics are entered, which include, for example, the individual's or client's: weight, height, age, gender, activity, and metabolic rate. The characteristics can also include: body fat percentage, body mass index, VO2 assimilation rate, lactic acid assimilation rate, and sleep schedule. When completed, program execution returns to segment 499.

Additional personal characteristics include, but are not limited to: health factors, anthropometrics and lab values. Health factors include, for example, diabetes, high cholesterol, etc. Anthropometrics are objective measurements of the individual's body. For example, an individual's or client's anthropometrics include, but are not limited to circumference measurement for, waist, hips, upper arm, thigh, lower leg, neck, and chest. Lab values are measurements of an individual's or client's physiological process through a lab test. An individual's or client's lab values include, for example, iron level, and oxygen level.

Once a profile is created, the program 301 saves the individual or client profile to a storage area of the program 10 for later recall and use, e.g., the individuals database. Additional individual's or client's information entered at a subsequent time is automatically appended to the individual's or client's profile, respectively. For example, an individual's or client's weight is entered into the program 301 on different days and each of the weights is stored as part of the individual's or client's profile, respectively. Thus, the individual's or client's weight can be tracked over time. In an approach, the individual's or client's data is stored as a record and updates to the individual's or client's data is either appended to the original data or stored as a separate record. Each modification to a profile includes the date of the modification. In an aspect, program 301 tracks the high, low and current values for each of the data values. For example, not only can a professional, a client, or an individual view his high, low, and current values for his cholesterol, but the program 10 accesses the historical records and displays his cholesterol during a designated time period.

From segment 499, an individual or client can select food preferences 403 and the program 301 execution will continue to this program segment. This segment of the program 301 enables the individual or client to tailor his food selection. This food selection is based on two aspects: First, the individual or client adding new food items or recipes to the food database. Second, the individual or client can specify her/his preferred categories of food to be searched. Food categories include, for example: accompaniments, baby foods, beef, beverages, bread, cereals (ready to eat), cereal and grain products, cookies & crackers, dairy products, desserts, entrees, fats and oils, finfish and shellfish products, fruits, ingredients, lamb and veal, legumes, medical, nuts and seeds, pork, poultry, restaurant menu items, fast food, restaurant menu items. generic, sausages and lunch meats, side dishes, snacks, soup, spices, sport and diet nutritionals, vegetables, vitamin and mineral supplements, and commercial ingredients.

When adding a food item, the individual or client enters name of the food, the food's base service measurement (e.g., 1 cup), a category of food that the food belongs to and nutritional information for the food item for the service size (e.g., the number of: calories, carbohydrates, fats, proteins). More than one category can be included, in an exemplary approach; up to three categories are permitted. Once the individual or client has entered all of the information and has accepted it, the food is added to the food database. Additionally, there is a data entry made into the individual's or client's profile indicating that this individual or client added this food. An individual or client can also add a new recipe and provide the constituent foods, the amount of each of the constituents foods, the nutritional components for each the foods, and the total of each of the nutritional components for the recipe. Once the individual or client has entered all of the information and has accepted it, the recipe is added to the food database. Additionally, there is a data entry made into the individual or client data record indicating that this individual or client entered this recipe.

An individual's or client's selection of preferred food categories. As noted above, each food item in the food database (FIG. 4) belongs to a category. An individual or client can select categories of food to search within for planning meals. More than one category can be searched. If a professional identifies a client as the member of a group or community, which is noted in the client's profile, then the client can search the food database by a group a community name.

From segment 499, an individual or client can select energy expenditure 404 and the program 10 execution will continue to this program segment. In an Energy Expenditure Calculation segment 404 an individual or client enters his energy expenditure for a period(s) of time. In an exemplar aspect, the period of time is a day (i.e., a 24 hour period). An individual or client may enter more than period's worth of energy expenditure. Generally, energy expenditure information entered in advance of the activity is forecasted, projected, or estimated energy expenditure. Generally, energy expenditure information entered after the activity is the historic or retrospective energy expenditure. In an aspect, an individual's or client's energy expenditure is entered on more than one occasion so that a series of data for an individual or client is stored, which, in an aspect, can be used as historical data, which can be used for analysis at a subsequent time.

In an aspect, the program 301's nutritional analysis incorporates the energy expended by the individual or client. In a general description, for example, the individual or client categorizes his daily energy expenditure into one of five categories based on the person's perspective of his weekly activities: (1) “Little to no exercise”; (2) “Light exercise (1-3 days per week)”; (3) “Moderate exercise (3-5 days per week)”; (4) “Heavy exercise (6-7 days per week)”; and (5) “Very heavy exercise (twice per day, heavy workouts).” Based on the category chosen, an individual' or client's daily energy expenditure is reasonably estimated, as seen for example below in Table A:

TABLE A

| Category | Description | Estimated Energy Expenditure |
|---|---|---|
| (1) | “Little to no exercise” | RMR × 1.2 |
| (2) | “Light exercise (1-3 days per week)” | RMR × 1.375 |
| (3) | “Moderate exercise (3-5 days per week)” | RMR × 1.55 |
| (4) | “Heavy exercise (6-7 days per week)” | RMR × 1.725 |
| (5) | “Very heavy exercise (twice per day, heavy workouts).” | RMR × 1.9 |

In Table A, RMR refers to Resting Metabolic Rate. Thus, for example if it is determined that a person has a RMR of 1994 and the person indicates that his energy expenditure is a Category (3) “Moderate Exercise”, where the category factor is 1.55, then the person's daily energy expenditure is estimated to be:

RMR×1.55=1994×1.55=3091 Kcal/day (1)

In Equation (1), RMR, which is a baseline, and is described below, is used as a reference to compare rest to a person's activities. In an aspect, a Category (0) is applicable to when the person is resting or sleeping, as such his Estimated Energy requirement is his RMR.

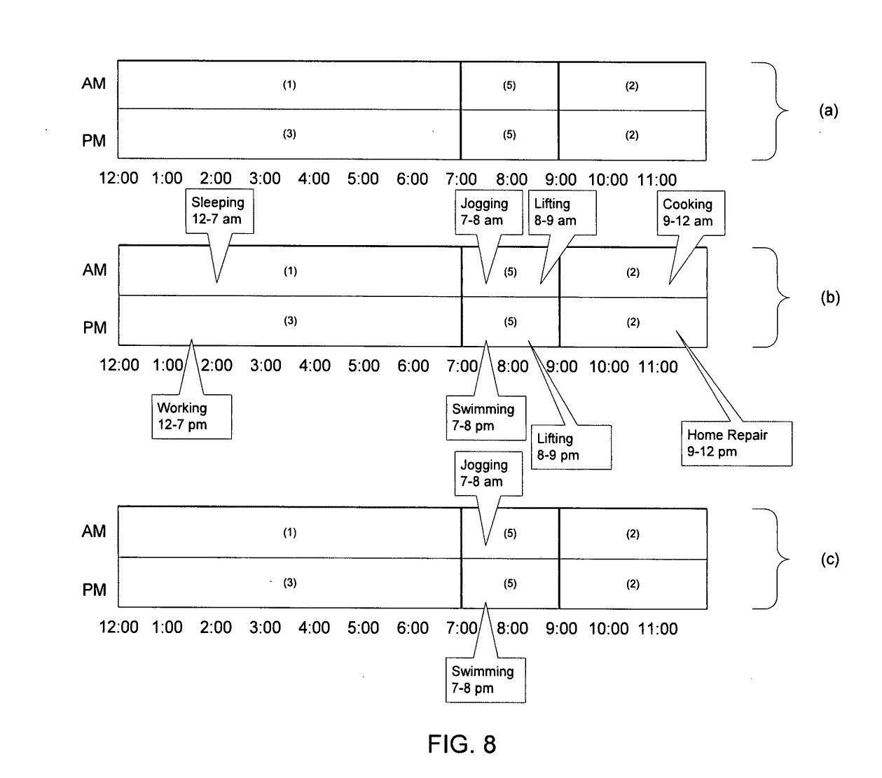

In an alternative approach, an individual or client can enter a more detailed description of forecasting his estimated energy expenditure. In this manner, the individual or client designates the length of time doing different types of activities. For example, the individual or client is provided a 24 hour day table, and the individual or client describes or labels segments of the day table according to his expected activity. The description can be generic, as referenced above. For example, FIG. 8(a) depicts a graphical representation of a 24 hour day, where an individual or client, as stepped through by the software program 301 has provided more a descriptive indication of his daily activities. As depicted in FIG. 8(a), the individual or client has indicated that he expects that: from 12:00 am to 7:00 am he will be doing a category (1) activity; from 7:00 am to 9:00 am he will be doing a category (5) activity; from 9:00 am to 12:00 pm he will be doing a category (2) activity; from 12:00 pm to 7:00 pm he will be doing a category (3) activity; from 7:00 pm to 9:00 pm he will be doing a category (5) activity; and from 9:00 pm to 12:00 am he will be doing a category (2) activity. Thus, the energy expenditure for the day is estimated to be the portion of the day spent at the activity×RMR×category factor and is calculated as:

7/24×RMR×Category (1)+2/24×RMR×Category (5)+3/24×RMR×Category (2)+7/24×RMR×Category (3)+2/24×RMR×Category (5)+3/24×RMR×Category (2)= (2)

7/24×(RMR×1.2)+2/24×(RMR×1.9)+3/24×(RMR×1.375)+7/24×(RMR×1.55)+2/24×(RMR×1.9)+3/24×(RMR×1.375)= (3)

7/24×(1994×1.2)+2/24×(1994×1.9)+3/24×(1994×1.375)+7/24×(1994×1.55)+2/24×(1994×1.9)+3/24×(1994×1.375)≈2916 Kcal (4)

Thus, the individual's or client's estimated energy expenditure for the day is approximately 2916 Kcal.

In another aspect, the individual or client desires to add more description to his estimated day. See, for example, FIG. 8(b). Instead of using general categories, the person enters an activity, preferably an activity in the activity database, described above with reference to FIG. 6, which is more descriptive of the activity, e.g., bicycling, and the time performing that activity. In an exemplary program 301, the program predefines a series of at least 900 activities with corresponding expected energy expenditure factors. An activity may have variations. For example, there are different variations of bicycling: bicycling, 10-11.9 mph, leisure, slow, light effort; bicycling, 12-13.9 mph, leisure, moderate effort; bicycling, 14-15.9 mph, racing or leisure, fast, vigorous effort; bicycling, 16-19 mph, racing/not drafting or >19 mph drafting, very fast; and bicycling, >20 mph, racing, not drafting. Each of these variations of bicycling has different estimated energy expenditure. For example, as seen below in Table B, each of the seven different variations of bicycling has a corresponding energy expenditure factor, i.e., its corresponding MET:

TABLE B

| MET | ACTIVITY |
|---|---|
| 4.0 | bicycling, <10 mph, leisure, to work or for pleasure |
| 8.0 | bicycling, general |
| 6.0 | bicycling, 10-11.9 mph, leisure, slow, light effort |
| 8.0 | bicycling, 12-13.9 mph, leisure, moderate effort |
| 10.0 | bicycling, 14-15.9 mph, racing or leisure, fast, vigorous effort |
| 12.0 | bicycling, 16-19 mph, racing/not drafting or >19 mph drafting, |
| | very fast |
| 16.0 | bicycling, >20 mph, racing, not drafting |

Each record of an individual's or client's estimated daily energy expenditure is saved in a data file for use at a later time, for example, to track the frequency of a particular activity. Although the program 301 utilizes a predefined list of activities found in the activities database 482, the activities can be added to the list, removed from the list, and the corresponding energy expenditure factor can be modified.

In another aspect, an individual or client creates a historical indication of the individual's or client's actual activities. As is commonly known, one's expectations for the day's schedule does not often reflect how the day is actually spent. Thus, in an aspect, contrary to the energy forecasting described above, an individual or client reflects back on a day and records the activity actually performed and the time spent doing the activity, thus providing a historical perspective his/her activities. In one approach, a copy of the individual's or client's estimated energy expenditure for a day is created, and an individual or client revises the forecasted record to reflect the historical reflection of the day's activities. For example, an individual projects a day, which includes having lunch from 12-2 p.m., walking from 2-4 pm, and performing household tasks from 4-6 pm. However, the weather on the day was extraordinarily beautiful, e.g., low humidity and moderate temperatures, and an individual changed his afternoon schedule which then included having lunch from 12-1 p.m., bicycling from 1-5 pm, and napping from 5-6 pm. Thus in one approach, in the creation of a historical record for a day, the individual modifies a copy of the estimated energy expenditure to reflect the actual activities performed. In another approach, a new energy expenditure record is created, in which the individual or client enters historical activity information.

An individual or client can also input his energy expenditure as it has been measured. For example, many commercial exercise machines have computers that provide readouts reflecting the individual's or client's workout. For example, a computerized treadmill typically will provide the time spent on the machine and will also provide its determination of the Mets or KCalories expended while on the machine. An individual or client records this number and enters it into the historical record of the day. If the individual or client works out on multiple machines or multiple times on the same machine the individual or client records the information from each of the machines and enters it into the historical record. In an aspect, the machines are networked to the program 301 and the individual's or client's data is uploaded to a networked computer system. For example, the corner gym's treadmills are connected to a central computer system. The computer system is connected, through the Internet, to the program 301 and the individual's or client's data from a treadmill can be transferred to the program 301.

In another aspect, energy expenditure is uploaded from a personal device that tracks energy consumption (i.e., calories burned), e.g., a calorimeter. For example, a Bodybugg™ is an electronic device that calculates an individual's energy consumption. Similarly, some Polar™ Heart Rate monitors provide an individual's energy consumption. In an aspect, the electronic device is networked to the program 301 and the individual's data is uploaded to the program 301.

In another aspect, an individual or client wears a calorimeter all day, and therefore the calorimeter calculates his energy consumption for the day. This data can be uploaded or entered into the program 301.

In another aspect, an individual or client desires to some description to his estimated day in addition to general descriptive. The individual or client can add provide addition description to one, some, or all of the day, to provide a somewhat detailed description. See, for example, FIG. 8(c). Program execution returns to segment 499.

From segment 499, an individual or client can select food intake 405 and the program 10 execution will continue to this program segment. In this segment, the individual or client records his historical food intake for a time period, e.g., a day. In an exemplary aspect, there are at least three meals (e.g., fuller meals) during the day and preferably several snacks (e.g., lighter meals). The individual or client steps through a food intake a meal/snack at a time. For each meal/snack the individual or client enters the time of the meal/snack, the name (e.g., breakfast, lunch, dinner, first snack, and second snack) of the meal/snack, and the name and quantity of the each food that was consumed during that meal/snack. When complete, program execution returns to segment 499.

From segment 499, an individual or client can select individual's goals 406 and the program 10 execution will continue to this program segment. In this segment, an individual or client selects a goal for her nutritional plan. For example, the individual or client chooses a fitness nutritional goal, which would include, for example, adjusting weight, body fat, or lean mass. The individual or client may alternatively choose a sport fitness nutritional goal, which would include, for example, training (e.g., in season, pre-season, post-season, off-season) and competition nutritional plans. For the competition nutritional plan, the individual enters the date of the competition. The individual's goal affect the nutritional make up of the individual's daily food intake. When completed, program execution returns to segment 499.

From segment 499, an individual or client can select summary 407 and the program 301 execution will continue to this program segment. In this segment, the program 301 displays a breakdown of the individual's or client's diet in terms of nutritional information, and may reflect, a high value, a low value and an average value for the nutritional information for designated period of time: For example, the program 301 displays, either as a singular or plurality, the following nutritional information of the individual or client: calories (expended), protein, carbohydrates (complex/starches, simple/sugars), dietary fat (total saturated fatty acids, total monounsaturated fatty acids, total polyunsaturated fatty acids, cholesterol), calories (consumed), total fiber, minerals (calcium, iron, magnesium, phosphorus, potassium, sodium, zinc, copper, manganese, selenium), vitamins (vitamin a (iu), alpha-carotene, beta-carotene, beta-cryptoxanthin, c, e, k, thiamin, riboflavin, niacin, pantothenic acid, vitamin b-6, vitamin b-12, choline (total)), dietary folate equivalents (DFE), carotenoids (Lycopene, Lutein+zeaxanthin).

The program 301 may also display the nutritional information as it pertains to the individual's or client's goals. For example, for a user seeking weight loss and providing the program 301 with a time period, the program 301 displays the user's weight and caloric intake over the time period.

When the professional uses the summary function for a team, the nutrition programming functionality for team sports nutrition analyzes all of the clients of a team individually and provides the professional with the summary nutrition information as to a summary of all of the team members over a time period. For example, the summary function provides the professional with the average caloric intake of team members during the month of March. When completed, program execution returns to segment 499.

When the professional uses the summary function for a team, the nutrition programming functionality for team sports nutrition analyzes all of the clients of a team individually and provides the professional with the average nutrition recommendation values that can be applied accurately to the team members. Because a large error can occur if the nutrition needs of the individuals are too broad, a warning appears for the user if there is a more than 10% variation in the nutrition needs. This warns the user that the recommendations are less accurate but it is ultimately up to the user if they want to proceed with the team recommendations.

From segment 499, a professional can select exercise prescription 408 and the program 10 execution will continue to this program segment. Prior creating the actual meal plan, the professional has the option to view and make modifications to a client's exercise or activity plan. This modified exercise or activity plan is provided to the client with the meal plans so that they have a detailed description of any changes that professional wants to make to their exercise or sport activities. The changes that are made are used in the calculation of the client's recommended daily nutritional input. For example, if the professional adds an hour of training in the afternoon, this would initiate pre, during, and post exercise nutrition recommendations for that time frame for the individual as they are creating their meal plan. This is important as the meal plan's nutrition information is not dictated by that the individual was doing, but more with what the individual will be doing during the time the individual will be following the nutrition plans provided to them. This increases the effectiveness and accuracy of the system.

In this segment, the professional views the client's exercise and activity plan, similar to that described above with respect to the client's ability to create and modify an estimated exercise and activity plan. The professional selects an activity(ies) to be modified and modifies the activity to selected activity. For example, a professional reviews a client's exercise and activity plan and notes that the client has light exercise scheduled from noon to 6 pm. The professional, wanting to increase the energy expenditure for the day, selects the light exercise activity and modifies it to last from noon to 2 pm. The professional then adds two additional exercises: a vigorous bicycle ride from 2 pm to 4 pm and a light exercise activity from 4 pm to 6 pm. In a preferred embodiment, after the professional saves the modified exercise and activity plan, a signal is provided to the client indicating that a change has occurred in the exercise and activity for the modified day. The signal is, for example, an email or text message. A signal is also provided when the individual accesses the system. For example, the button reflecting the energy expenditure is a different color or flashes to reflect that the client to take a look at the exercise and activity plan to see what changes have occurred. In an aspect, the modified activities in the client's exercise and activity plan are highlighted to draw the client's attention to which activities have been modified. Alternatively, the modified exercise plan is emailed to the client. When completed, program execution returns to segment 499.

From segment 499, a professional or individual can select meal planning and the program 301 execution will continue to this program segment 409. In this segment, the professional or individual views the client's or individual's nutritional goal and their estimated energy expenditure, if provided, to create a meal plan.

After an individual or client has entered his characteristics, the individual or professional can plan meals for the individual or client, respectively. The basis for meal planning is calculating the number of calories required per day for a person. For example, a typical male's daily caloric requirement can be determined by averaging two different formulas:

MF1=10*Weight (kg)+6.25*Height (cm)−5*age (years)+5 (5)

MF2=66.47+6.23*weight (lbs)+12.67*Height (Inches)−6.76*age (years) (6)

By averaging the results of equation (5) and (6), a typical male's daily caloric requirement is found. Thus:

A typical male's daily caloric requirement=(MF1+MF2)/2 (7)

A female typically has a different metabolic process and therefore her caloric needs are determined differently:

10*Weight (kg)+6.25*Height (cm)−5*age (years)−161 (8)

655.1+4.34*weight (lbs)+4.69*Height (Inches)−4.68*age (years) (9)

By averaging the results of equation (8) and (9), a typical female's daily caloric requirement is found. Thus:

A typical female's daily caloric requirement=(MF1+MF2)/2 (10)

Meal planning depends on a nutritional goal ratio of carbohydrates, fats, and protein, as expressed as a percentage of the daily caloric intake. In the exemplary approach, if no nutritional goal ratio is selected, the a default nutritional goal ratio is applied, for example, carbohydrates comprise 55% of daily caloric intake, proteins comprise 23% of daily caloric intake, and fats comprise 22% of daily caloric intake. This nutritional goal ratio can be altered by an individual or client. For example, the individual or client desires more focus on proteins, then nutritional goal ratio could be where: carbohydrates comprise 45% of daily caloric intake, proteins comprise 35% of daily caloric intake, and fats comprise 20% of daily caloric intake. In another approach, the individual or client desires more focus on carbohydrates, then nutritional goal ratio could be where: carbohydrates comprise 60% of daily caloric intake, proteins comprise 20% of daily caloric intake, and fats comprise 20% of daily caloric intake.

If the individual or client has not entered a nutritional goal nor an estimated expended energy for the day, then the program 301 uses system defined default values as the recommended nutritional values for the day. For example, nutritional guidelines provide an estimate daily nutritional intake, specified, by gender and age. These guidelines not only include a recommended caloric intake to maintain current weight but also recommends a percentage of or amount of nutritional components. For example, 55% of the recommended caloric intake should come from proteins, 23% of the recommended caloric intake should come from carbohydrates, and 22% of the recommended caloric intake should come from fats.

If the individual or client has entered a nutritional goal but has not entered an estimated expended energy for the day, then the system uses defined values based on the nutritional goal and the characteristics as the recommended nutritional values for the day. For example, nutritional guidelines provide an estimate daily nutritional intake, specified, by gender, age, and nutritional goal. If the goal is to lose weight, then for example, there is a recommended caloric intake K2, 55% of the recommended caloric intake should come from proteins, 23% of the recommended caloric intake should come from carbohydrates, and 22% of the recommended caloric intake should come from fats, where K2 is determined to be a value less than the determined daily caloric requirement. (see, for example, Equation (10) above). If the fitness goal is to lose weight, then the meal planning recommends less calories per day then the person's daily caloric requirement. The difference in calories effects the rate of weight loss. If the fitness goal is to lose body fat, then the meal planning recommends a small amount of fat, e.g., less fats per day then the person's recommended daily intake of fats. The decrease in fat intake effects the rate of fat loss.

If the individual or client has entered a nutritional goal and an estimated expended energy for the day, then the system uses defined values based on the nutritional goal and the characteristics as the recommended nutritional values for the day with an adjustment for the estimated energy expenditure. If the goal is to lose weight, then for example, there is a recommended caloric intake K3, 55% of the recommended caloric intake should come from proteins, 23% of the recommended caloric intake should come from carbohydrates, and 22% of the recommended caloric intake should come from fats. K3 is an amount of calories that will compensate for estimated expended energy for the day.

If the individual or client has entered estimated expended energy for the day, which includes a significant event, e.g., competition, training exercise or vigorous activity, the program 301 will recommend as part of the meal planning of the day for the individual or client a snack(s) at a specified time period before, after and during the significant event. The recommended snack will also include recommended nutritional values for the snack. In a preferred approach, the time period is 1 hour before and after the event. 50% of the estimated calories expended during the event are recommended to be consumed by the individual or user within one hour after the event occurs. A second percentage 10% of the estimated calories expended during the event are recommended to be consumed by the individual or user within one hour preceding the event. A third percentage 20% of the estimated calories expended during the event are recommended to be consumed by the individual or user within during the event. The Exemplary recommended value nutritional values are determined by the program 301. The program 301 determines the individual's or user's recommended daily nutritional intake for the day and subtracts out the calories consumed by the individual or user before, during and after the event. The remaining nutritional intake amounts are used as part of the meal planning, to determine the nutritional intake for each meal.

A professional or system administrator defines a default number of preferred meals per day and snacks per day, e.g. 3 meals (breakfast, lunch, dinner) and 2 snacks (e.g., mid morning snack, and mid afternoon snack). A professional or system administrator can modify the preferred number of meals per day and snacks per day, for a client, an individual, a team, or for all users of the system 10.

In an exemplary aspect, as the professional or individual is planning the meal for the client or himself, respectively, the program 301 displays on the computer display a summary of the client's or individual's estimated nutritional food intake for the day, broken down by meal/snack. The program 301 also displays a summary of the client's or individual's estimated nutritional food intake for each meal/snack, and providing selected nutritional values for the each of the food items comprising the meal/snack. Also displayed for the client or individual is the client's or individual's recommended food intake for the day, broken down by meal/snack, and providing selected nutritional values for the meals/snacks.

The program guides the individual or professional through planning each meal/snack. For each meal/snack the professional or individual enters the time of the meal/snack, the name (e.g., breakfast, lunch, dinner, first snack, and second snack) of the meal/snack, and then selects food to be consumed during the meal. In an aspect, the professional or individual selects foods, and their quantity, preferably from client's or individual's preferred food category. The professional or individual can also select food from the general database. It is desirable to choose foods for the meal/snack that will meet or come close to the recommended nutritional approach for the meal.

In an exemplary approach, daily caloric intake is roughly divided over the meals of the day. For example, if there three meals per day then each meal should have s ⅓ of the daily caloric intake, where the meals are consumed at times spread out through the day. If there are three meals and two snacks, e.g., mini-meals, per day then each meal should have approximately ¼ of the daily caloric intake, and each snack have approximately ⅛ of the daily caloric intake, where the meals/snacks are consumed at times spread out through the day. For example, if a client's recommended nutritional daily intake is 3000 Calories, 30 grams of carbohydrates, 15 grams of protein, and 6 grams of fat and if there will be three meals, then each of the three meals will roughly contain: 1000 Calories, 10 grams of carbohydrates, 5 grams of protein, and 2 grams of fat. In other aspects, the distribution is weighted depending on the meal. For example, breakfast would be 45% of the recommended daily nutritional intake values, thus lunch would be 35% of the recommended daily nutritional intake values, and dinner would be 20% of the recommended daily nutritional intake values.

Hydration is an intricate part of an individual's or client's nutritional plan. However, as water typically has not nutritional value, it is often overlooked as part of the nutritional plan. However, in an exemplary embodiment, fluids are included as part of the meal planning. As a guideline, it is recommended that an individual or client consume 1 Quart (32 oz) per 50 lbs bodyweight. Fluids, in generally, should be consumed throughout the day. In an aspect, if an individual's energy expenditure for the day includes a training exercise or a competition, then it is recommended that: 2 cups of fluid be consumed approximately 2 hours before the training exercise or competition, 1 cup of fluid be consumed approximately 0 to 1 hour before the training exercise or competition, and 2 cups of fluid be consumed approximately 2 hours after the training exercise or competition. The recommended amount of fluid consumed during the training exercise or competition depends on the exercise and the individual's goals.

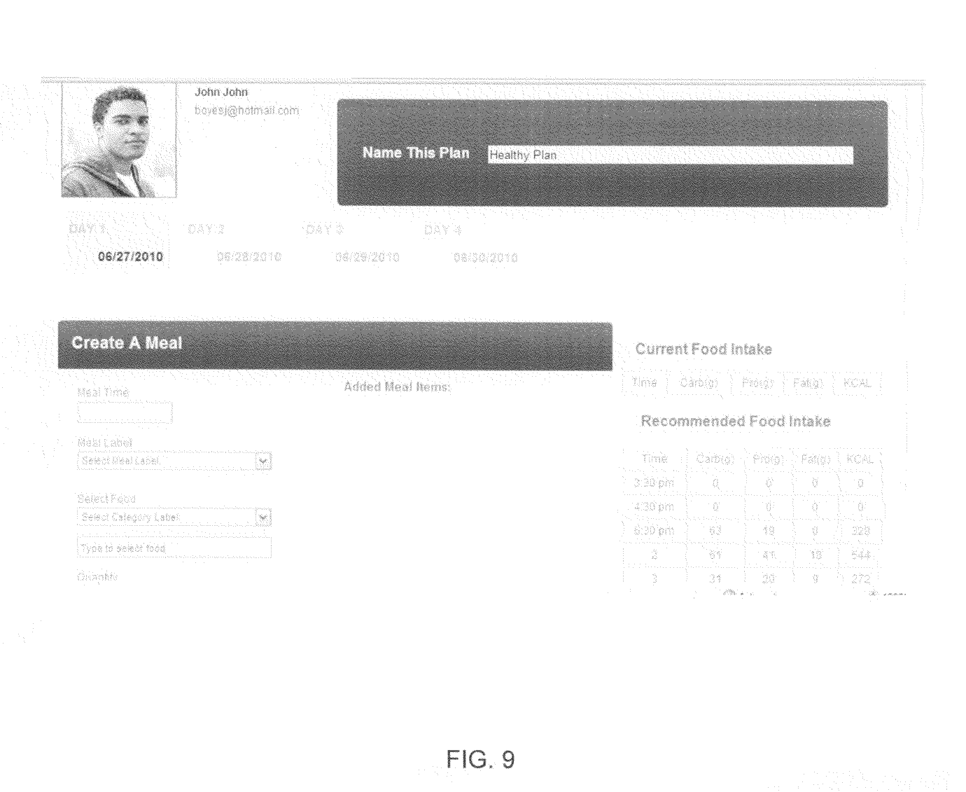

FIG. 9 depicts an exemplary screen display of a computer display running the computer system of FIG. 1. More specifically, FIG. 9 depicts a display as a program 301 running the meal planning component 409 for a selected day. Based on the goals and characteristics of the individual or client, the recommended nutritional intake for the day is displayed. The program suggests the recommended nutritional intake for three meals at suggested times during the day. If the individual or client has an estimated energy expenditure record or an exercise prescription for the day, the program displays the recommended nutritional intake for different meals and snacks at suggested times during the day. The user selects the meal time, the meal label, the food and the food quantity for each meal. As the user selects food the program displays the current food intake, which permits the user to compare the current food intake to the recommended nutritional intake for the day. A user can modify meals and food items.

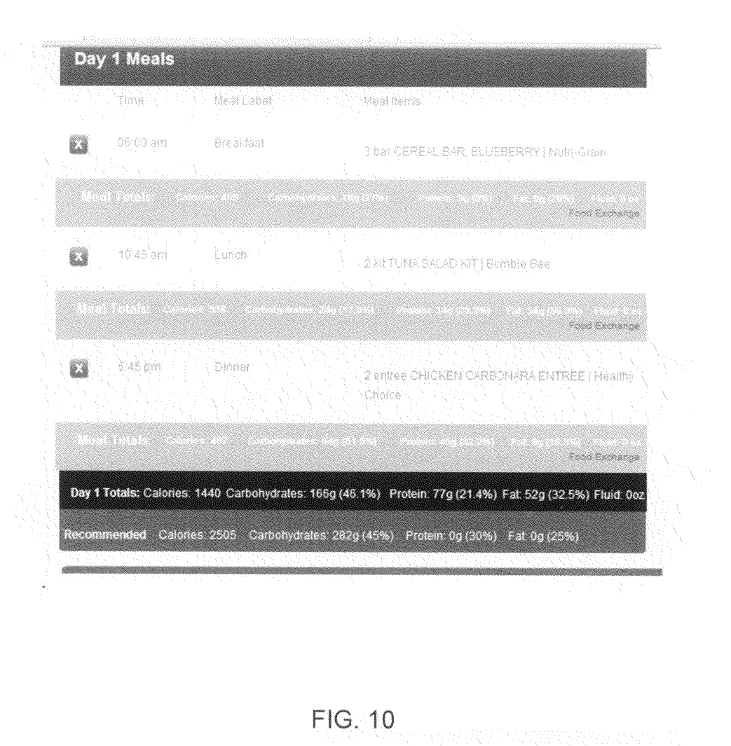

When the user has finished planning the meal for the day, the meal plan is displayed. FIG. 10 depicts a completed meal plan for a day. As seen in FIG. 10, the program displays three meals, breakfast, lunch and dinner. For each meal, the program displays, the time of the meal, the food that comprises the meal, and the nutritional intake for the meal, e.g., Calories, Carbohydrates (in terms of total amount in grams and percentage as it relates to total calories), Protein (in terms of total amount in grams and percentage as it relates to total calories), fats (in terms of total amount in grams and percentage as it relates to total calories), and fluid. The user can accept the meal plan and return program execution to segment 499. The user can also modify the meal plan and return program execution to segment 499.

A professional can also plan meals for clients who are members of the team. For example, the professional uses combined values of personal characteristics of the members of the team and uses those combined values to determine the recommended daily nutritional values. The professional then plans the team's meals based on the recommended daily nutritional values.

Because a large error can occur if the nutritional needs of the individual clients of a team are too broad, a warning appears for the professional if there is a more than 10% variation in the nutrition needs. This warns the professional that the recommendations are less accurate but it is ultimately up to the professional if they want to proceed with the team recommendations. When completed with meal planning, program execution returns to segment 499.

In the images segment 410, an individual or client can upload of images of themselves to the program 301 to be stored in their profile. Images of the individual are maintained in the system so that the individual's physical changes can be monitored.

In analysis segment 412, a professional or individual can analyze different data. A professional can analyze a client's or team's data and an individual can only analyze her/his own data. Statistical Analysis: The system is designed for the input and analysis of data. This is done at an indirect analysis level by the professional or individual, where they interpret the results of the data entered or calculated. A more precise method for analysis will be to incorporate statistical analysis into functions of the programming. More specifically a within subject design will be used that allows for the analysis of multiple people over multiple trials. A multiple regression analysis will be used for this design that allows the user to explore the relationships between variables, such as the relationship between a change in health or sports characteristics and a nutrition component (i.e., calories, protein, carbohydrates . . . ). This would allow the professional or individual to view a causal relationship between variables and provide definitive evidence that a specific factor of nutrition affected another health or sports variable. This is critical for the direct application of the system in professional settings, as a direct cause and effect relationship will be identified.

In statistics, regression analysis includes any techniques for modeling and analyzing several variables, when the focus is on the relationship between a dependent variable and one or more independent variables. More specifically, regression analysis helps us understand how the typical value of the dependent variable changes when any one of the independent variables is varied, while the other independent variables are held fixed. Most commonly, regression analysis estimates the conditional expectation of the dependent variable given the independent variables—that is, the average value of the dependent variable when the independent variables are held fixed. Less commonly, the focus is on a quartile, or other location parameter of the conditional distribution of the dependent variable given the independent variables. In all cases, the estimation target is a function of the independent variables called the regression function. In regression analysis, it is also of interest to characterize the variation of the dependent variable around the regression function, which can be described by a probability distribution. Regression analysis using multiple regression models would allow the user to see positive or negative correlations between multiple factors of nutrition, health, or performance.

Thus, a professional or individual designates factors to be analyzed through multiple regression analysis. In an exemplary approach, the professional or individual analyzes a performance value against at least one nutritional component. Thus, the professional or individual compares the recorded, historical values for an client's or individual's performance value (e.g., the time to complete a one hundred meter course) versus the intake of carbohydrates.

Additionally, when an individual or client profile is created, a specific sport can be selected to be associated with that individual or client which enables the professional to compare and analyze multiple profiles within a given sport. When completed, program execution returns to segment 499.

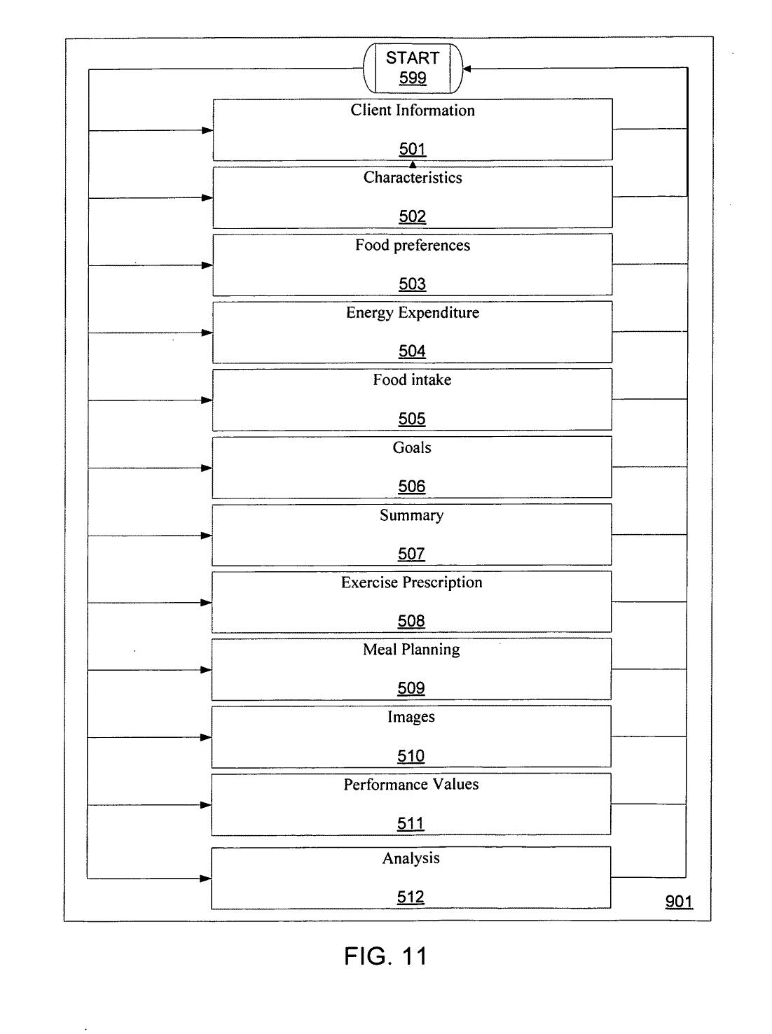

In another aspect, the user selects sports fitness nutrition program 901 instead of standard nutrition program 301, which includes one additional program segment—performance values 511 as depicted in FIG. 11. Other than described below, program segments of program 901: client information 501, characteristics 502, food preferences 503, energy expenditure 504, food intake 505, goals 506, summary 507, exercise prescription 508, meal planning 509, images 510, and analysis 511 operate similarly to their comparable name program segments of program 301: client information 401, characteristics 402, food preferences 403, energy expenditure 404, food intake 405, goals 406, summary 407, exercise prescription 408, meal planning 409, images 410, and analysis 411.

The performance values segment 511 enables the input and tracking of any performance factor or feat; such as weight lifting (e.g., bench press), speed measurements (e.g., 40 yd dash), agility work (e.g., cone drills), and any other feat the professional wants to test and track/compare with the nutrition intake of the individual or team. In this program segment, a user records, from time to time, at least one performance factor which is recorded in the profile of the user or individual. Subsequent performance factor entries are appended to the profile of the user or individual. Thus, a historical record is created of the performance factor. When completed, program execution returns to segment 599.

Sports fitness nutrition program 901 is more detailed with regards to calculating nutrition needs of the athlete in the time frame before and after training or competition. The standard fitness program 301 determines nutrition needs shortly before and shortly after the event, for example, 1 hour pre and post exercise nutritional needs. The Sports fitness nutrition programming 901 determines nutrition needs at longer time periods before and after the event, for example, several hours pre and post exercise nutritional needs. Example: 4 to 8 hours before and after an event or practice instead of just 1 hour. The user/professional, prior to creating meal plan, can identify in the sports nutrition section the competition date and time. This will allow the program to calculate nutritional preparation in the hours and days preceding an event. In certain nutritional protocols, nutrition is planned several days before an athletic event. For example, determining a carbohydrate loading protocol for an endurance or high-endurance event in order to most effectively prepare for the competition.

An additional feature of sports nutrition program 901 is the ability to select training types: “endurance training and endurance competition” and “strength endurance and strength competition. These training types can be further specified with additional fitness nutritional goals, e.g., adjust body weight, adjust body fat, etc. The program 901 determines recommended nutritional intakes for a day and for meals/snacks within the day.

For example, if an individual or client selects “endurance training and endurance competition” training without selecting a fitness nutritional goal, then the program 901 determines that the recommended daily nutritional intake for endurance training is: carbohydrates being 65% of total calories, protein 15% of total calories and fat 20% of total calories. For endurance competition, program 901 determines that the recommended daily nutritional intake is: carbohydrates being 60% of total calories, protein 20% of total calories and fat 20% of total calories. Program 901 determines that the recommended nutritional intake for meals around the training exercise to be: for a snack 1 hour before the training exercise: carbohydrates 1 g/kg/bw and protein 0.2 g/kg/bw; for a snack 0-1 hour before the training exercise: carbohydrates 0.5 g/kg/bw and protein 0.3 g/kg/bw; for a snack 0-1 hour after the training exercise: carbohydrates 1 g/kg/bw and protein 0.3 g/kg/bw; and for a snack 2 hours after the training exercise: carbohydrates 2 g/kg/bw and protein 0.5 g/kg/bw, where bw is body weight.

If the individual has a completed “Energy Expenditure”/“Exercise Prescription” as part of his profile for the day when meals are being planned, then the program 901 determines the nutrition during the training exercise to be: fluid (oz) is calories expended/22.4 and carbohydrates calories expended/16. The program 901 also modifies the snacks for 0-1 hour after the training exercise to be: carbohydrates=(calories expended×0.5)/4 and protein=(calories expended×0.125)/4. Calculation of total calories is a combination of calculating baseline calorie needs over 24 hours (0092 above) and a multiple of this value as determined by the individual's or client's activity level as noted above.

In another example, if an individual or client selects “endurance training and endurance competition” training and also selects a fitness nutritional goal of weight loss, then the program 901 determines that the recommended daily nutritional intake for endurance training is: carbohydrates being 65% of total calories, protein 15% of total calories and fat 20% of total calories. For endurance competition, program 901 determines that the recommended daily nutritional intake is: carbohydrates being 60% of total calories, protein 20% of total calories and fat 20% of total calories. Program 901 determines that the recommended nutritional intake for meals around the training exercise to be: for a snack 1 hour before the training exercise: carbohydrates 0.5 g/kg/bw and protein 0.2 g/kg/bw; for a snack 0-1 hour before the training exercise: water; for a snack 0-1 hour after the training exercise: carbohydrates 0.5 g/kg/bw and protein 0.15 g/kg/bw; and for a snack 2 hours after the training exercise: carbohydrates 1 g/kg/bw and protein 0.5 g/kg/bw.

If the individual has a completed “Energy Expenditure”/“Exercise Prescription” as part of his profile for the day when meals are being planned, then the program 901 determines the nutrition during the training exercise to be: fluid (oz) is calories expended/22.4 and a low calorie electrolyte replacement drink. The program 901 also modifies the snacks for 0-1 hour after the training exercise to be: carbohydrates=(calories expended×0.5)/4 and protein=(calories expended×0.15)/4.

For example, if an individual or client selects “strength training and strength competition” training without selecting a fitness nutritional goal, then the program 901 determines that the recommended daily nutritional intake for strength training is: carbohydrates being 65% of total calories, protein 15% of total calories and fat 20% of total calories. For strength competition, program 901 determines that the recommended daily nutritional intake is: carbohydrates being 60% of total calories, protein 20% of total calories and fat 20% of total calories. Program 901 determines that the recommended nutritional intake for meals around the training exercise to be: for a snack 1 hour before the training exercise: carbohydrates 1 g/kg/bw and protein 0.3 g/kg/bw; for a snack 0-1 hour before the training exercise: carbohydrates 0.5 g/kg/bw and protein 0.3 g/kg/bw; for a snack 0-1 hour after the training exercise: carbohydrates 1 g/kg/bw and protein 0.4 g/kg/bw; and for a snack 2 hours after the training exercise: carbohydrates 2 g/kg/bw and protein 0.5 g/kg/bw.

If the individual has a completed “Energy Expenditure”/“Exercise Prescription” as part of his profile for the day when meals are being planned, then the program 901 determines the nutrition during the training exercise to be: fluid (oz) is calories expended/22.4 and carbohydrates calories expended/16. The program 901 also modifies the snacks for 0-1 hour after the training exercise to be: carbohydrates=(calories expended×0.5)/4 and protein=(calories expended×0.125)/4.

For example, if an individual or client selects “strength training and strength competition” training and selects a fitness nutritional goal of weight loss, then the program 901 determines that the recommended daily nutritional intake for strength training is: carbohydrates being 65% of total calories, protein 15% of total calories and fat 20% of total calories. For strength competition, program 901 determines that the recommended daily nutritional intake is: carbohydrates being 60% of total calories, protein 20% of total calories and fat 20% of total calories. Program 901 determines that the recommended nutritional intake for meals around the training exercise to be: for a snack 1 hour before the training exercise: carbohydrates 0.5 g/kg/bw and protein 0.3 g/kg/bw; for a snack 0-1 hour before the training exercise: water; for a snack 0-1 hour after the training exercise: carbohydrates 0.5 g/kg/bw and protein 0.3 g/kg/bw; and for a snack 2 hours after the training exercise: carbohydrates 1 g/kg/bw and protein 0.5 g/kg/bw.

If the individual has a completed “Energy Expenditure”/“Exercise Prescription” as part of his profile for the day when meals are being planned, then the program 901 determines the nutrition during the training exercise to be: fluid (oz) is calories expended/22.4 and low calorie electrolyte replacement drink. The program 901 also modifies the snacks for 0-1 hour after the training exercise to be: carbohydrates=(calories expended×0.5)/4 and protein=(calories expended×0.15)/4.

Thus, the program 901 takes into consideration the client's or individual's training and any fitness nutritional and provides food intake recommendations at tie periods before and after training or competition.

The standard fitness program segment 301 and the sports fitness nutrition program segment 901 share the databases depicted in FIG. 4. An individual's or client's profile in the individuals database 488, food database 480, activity database 482, DRI database 484, and professionals database 486 can be accessed from both the standard fitness program segment 301 and the sports fitness nutrition program segment 901. Any modifications to a database is accessible and can be utilized by the standard fitness program segment 301 and the sports fitness nutrition program segment 901.

In both the sports fitness nutrition program 901, and the standard fitness nutrition program 301 a professional has access to all program functions as an individual. Thus, the professional can enter data as a client or she can enter data on behalf of an individual, where the data is stored in the individuals database. The professional also has additional function: she administers individual's records (e.g., creating and modifying an individual's records), administers team's records (e.g., creating and modifying team's records), analyze an individual's and team data). The professional can also prescribe exercise and activity plans for individuals and teams as well as the professional can prescribe meal plans.

Administration. As part of the administration of a client or team, the professional can request information from client. For example, the professional sends a client an information request through a web link or an email. The information request can be, for example, a request for the client's food intake for a specified day(s) or the client's energy expenditure for a specified day(s). If the information request is sent in the form of a link, then the client, upon selecting the link, is connected to the program 301 or program 901, and, more specifically, to a relevant segment of program 301 or 901 and provided a template of the information requested to enable the client to enter information as part of the program 301 or program 901. However, the client does not need to sign in or provide any access information to access, and run program 301 or 901; the link sent by the professional to the client provides that information to the program 301 or program 901. Once the client has completed the information template, the information provided by the client is automatically added to the client's profile in the individuals database.

If the professional sends an information request to a client in an email, an information template is provided which indicates what information the professional is seeking, e.g., food intake or energy expenditure. The information template is either attached to the email or part of the email. The client enters data into the template and sends the email with the completed information template preferably to an e-mail address associated with the program 301 or 901. The program 301 opens the email and the information provided by the client is automatically added to the client's profile in the individuals database. The information template and the email are independent of the program 301 or program 901 and the client enters data into the template independently of program 301 or program 901. When the client sends back the email with the completed information template, the professional forwards the email, program receives the email, takes the record and appends it to the client's profile in the program 301.

This information request allows a professional to easily and quickly prompt a client for information and provides a client with a relatively quick and easy mechanism to provide the information to the professional through the program 301 or 901. Additionally, with an email information request, a client provides the information in a return email isolated from program 301 or 901 and does not access program 301 or 901.

When the client opens the information request she is prompted to provide information, where the information is, for example, the historical energy expenditure for a particular day or historical food intake for the day. The individual completes the information and returns the email or link which contains the information. The information is seamlessly incorporated into the program 10. Consequently, an individual remotely provides information to the program 10 without being networked into the program 10. Thus, the individual or professional can use the system as effectively if the individual is in the same room or if they are in another city or state.

Thus, at the conclusion of the sports fitness nutrition program 901 or the standard fitness nutrition program 301, the individual or client has a meal plan that helps the individual or client achieve his/her nutritional goal(s).

An exemplary embodiment of some of the features described above can be found in evolutionutrition™ software.

While the invention has been described and illustrated with reference to specific exemplary embodiments, it should be understood that many modifications and substitutions can be made without departing from the spirit and scope of the invention. For example, many of the action above are described with respect to an individual, and the invention is not so limited as a professional can act on behalf of an individual and perform the data entry for the individual. Accordingly, the invention is not to be considered as limited by the foregoing description but is only limited by the scope of the claims.

What is claimed as new and desired to be protected by Letters Patent of the United States is:

1. A processing system comprising:
a machine readable medium, said machine readable medium being encoded to control a processing device, whereby said processing device is adapted to plan a meal for a person, said meal meeting a nutritional need of said person, said processing device executing the steps of:
receiving biographical information about said person;
determining a daily energy expenditure of said person;
determining recommended daily nutritional intake for said person; and
planning a meal for said person using said biographical information, said energy expenditure, and said recommended daily nutritional intake.

2. The processing system of claim 1, wherein said step of determining an energy expenditure is based on receiving a general description of the person's lifestyle.

3. The processing system of claim 1, wherein said step of determining an energy expenditure is based on receiving a somewhat descriptive indication of the person's lifestyle.

4. The processing system of claim 1, further comprising:
receiving a food preference for the person,
wherein said step of planning a meal uses said person's food preference.

5. The processing system of claim 1, further comprising:
receiving exercise information for said person; and
planning a snack for the person using said exercise information, said biographical information, said energy expenditure, and said recommended daily nutritional intake.

6. The processing system of claim 1, further comprising:
receiving exercise information for said person; and
planning a plurality of snacks for the person using said exercise information, said biographical information, said energy expenditure, and said recommended daily nutritional intake, where one of said plurality of snacks occurs at a first time period before said exercise begins, where a second of said plurality of snacks occurs at a second time period after said exercise ends.

7. The processing system of claim 6, wherein said first and second time periods are approximately an hour.

8. The processing system of claim 1, further comprising:
receiving exercise information for said person; and
planning a plurality of snacks for the person using said exercise information, said biographical information, said energy expenditure, and said recommended daily nutritional intake, where one of said plurality of snacks occurs at a first time period before said exercise begins, where a second of said plurality of snacks occurs at a second time period after said exercise ends, where a third of said plurality of snacks occurs at a third time period before said exercise begins, where a fourth of said plurality of snacks occurs at a fourth time period after said exercise ends.

9. The processing system of claim 8, wherein said first and second time periods are approximately an hour, wherein said third and fourth time periods are approximately two hours.

10. The processing system of claim 1, wherein said meal planning incorporates the person's fitness goal.

11. The processing system of claim 10, wherein said person's fitness goal is weight loss.

12. The processing system of claim 1, further comprising:
sending an information request to a person;
receiving a modified information request from said person; and
incorporating data from said modified information request into a profile for said person.

13. The processing system of claim 12, wherein said step of sending an information request comprises sending an electronic link to the meal planning program.

14. The processing system of claim 12, wherein said step of sending an information request comprises sending an email to the person having an associated data template.

15. A computerized system comprising:
a processing device;
a communications device; and
a machine readable medium, said machine readable medium encoded to control said processing device such that said processing device is consequently adapted to determine a person's daily energy expenditure goal and said person's fitness goal and, thereafter, to select a food from a food database and add said food to a meal plan, said meal plan being configured to satisfy said energy expenditure goal and said fitness goal.

16. The computerized system of claim 15, wherein said processing device is further adapted to receive a new food to added to the food database.

17. The computerized system of claim 15, wherein said processing device is further adapted to receive a new food recipe to the food database.

18. The computerized system of claim 15, wherein said processing device is further adapted to receive an indication of a person's preferred category of food in the food database.

19. The computerized system of claim 15, wherein said act of selecting a food incorporates the person's preferred category of food as part of the selection process.

20. The computerized system of claim 15, wherein said processing device is further adapted to select food for a second meal of the day.
